(19) United States
(12) Patent Application Publication
(10) Pub. No.: US 2019/0209509 A1
(43) Pub. Date: Jul. 11, 2019

(54) Hepatoprotective potential of Calebin A

(71) Applicants: Muhammed Majeed, Edison, NJ (US); Kalyanam Nagabhushanam, East Windsor, NJ (US); Lakshmi Mundkur, Bangalore (IN)

(72) Inventors: Muhammed Majeed, Edison, NJ (US); Kalyanam Nagabhushanam, East Windsor, NJ (US); Lakshmi Mundkur, Bangalore (IN)

(21) Appl. No.: 16/238,677

(22) Filed: Jan. 3, 2019

Related U.S. Application Data
(60) Provisional application No. 62/613,843, filed on Jan. 5, 2018.

(51) Int. Cl. A61K 31/222; A61K 9/00; A61P 1/16; A61P 3/10

(52) U.S. Cl. CPC A61K 31/222; A61P 3/10; A61P 1/16; A61K 9/0053

(57) ABSTRACT

The invention discloses a novel use of Calebin A in the prevention of hepatocellular damage. More specifically, the invention discloses the use of Calebin A in mitigating hepatocellular damage in drug and alcohol induced liver toxicity. The invention further discloses a method for the management of hyperglycemia, associated with drug and alcohol induced liver damage by increasing glucose uptake using Calebin A or a composition comprising Calebin A.

### CROSS REFERENCE TO RELATED APPLICATION

This is a non-provisional US patent application claiming priority from U.S. Provisional application No. 62/613,843 filed on 5 Jan. 2018, the details of which are being incorporated herein by reference.

### BACKGROUND OF THE INVENTION

### Field of the Invention

The invention in general relates to composition comprising Calebin A. More specifically the present invention relates to hepatoprotective potential of compositions comprising Calebin A.

### Description of Prior Art

Liver is the primary organ involved in the metabolism of drugs, chemicals and alcoholic substances. Overdose of the above substances increases hepatocellular damage leading to the development of many liver diseases and disorders through different mechanisms such as inflammation, dysfunction of cytochrome P450, mitochondrial dysfunction and oxidative stress (Malaguarnera et al., Toxic hepatitis in occupational exposure to solvents, World J Gastroenterol., 2012; 18(22):2756-2766). Of the different factors responsible for liver damage, drug and alcohol induced hepatocellular damage garners much importance due to the increased frequency of drug and alcohol intake. According to a recent report by LiverTox (http://livertox.nlm.nih.gov), almost 535 of the drugs currently in use have a reported case history of liver injury.

Drug induced hepatotoxicity is the most frequent cause of acute liver failure. There are many pharmacological substances that cause liver damage which include anaesthetics, anticancer drugs, antibiotics, anti tuberculosis agents, antiretrovirals, and cardiac medications. Among the different drugs, Acetaminophen (Paracetamol) induced liver toxicity is the most common and widely studied.

The next most common type of hepatocellular damage is increased alcohol intake. Generally, alcohol is metabolized in the liver by the enzyme alcohol dehydrogenase (ADH), cytochrome P-4502E1 (CYP2E1); mitochondrial catalase. Enzymes CYP2E1 and ADH convert alcohol to acetaldehyde which triggers the hepatocellular deterioration (Bruha et al., Alcoholic liver disease, World Journal of Hepatology, 2012; 4(3):81-90). Excessive alcohol intake leads to many liver disorders such as Alcoholic liver disease (ALD), simple steatosis to cirrhosis and hepatocellular carcinoma.

Both drug and alcohol induced liver damage lead to secondary complications which include hyperglycemia, diabetes, hypercholesterolemia, and cardiovascular diseases.

There are many agents available in the market today for the treatment of drug and alcohol induced hepatotoxicity. Natural products rich in triterpene, flavonoids or polyphenols, are reported to act as powerful hepatoprotective agents (Singh et al., Drug-Induced Liver Toxicity and Prevention by Herbal Antioxidants: An Overview, Front Physiol., 2015; 6:1-18)

Curcumin, obtained from rhizomes of Curcuma longa exhibits many beneficial biological activities including antioxidant, anti-inflammatory, anticoagulant, antitumor activities. The hepatoprotective ability of curcumin with respect to drug and alcohol induced liver damage is reported in the following references:
1. Tung et al., Hepatoprotective effect of Phytosome Curcumin against paracetamol-induced liver toxicity in mice, Braz. J. Pharm. Sci., 2017; 53(1):1-13.
2. Naglaa et al., Protective Effect of Curcumin versus N-acetylcystein on Acetaminophen Induced Hepatotoxicity in Adult Albino Rats, J Cytol Histol, 2015, S3:018. doi:10.4172/2157-7099.S3-018
3. Manal et al., The protective effect of curcumin on paracetamol-induced liver damage in adult male rabbits: biochemical and histological studies, Egyptian Journal of Histology, 2014; 37(4):629-639.
4. Rang et al., Curcumin prevents chronic alcohol-induced liver disease involving decreasing ROS generation and enhancing antioxidative capacity, Phytomedicine, 2012; 19(6):545-50.
5. Xiang et al., Curcumin attenuates chronic ethanol-induced liver injury by inhibition of oxidative stress via mitogen-activated protein kinase/nuclear factor E2-related factor 2 pathway in mice, Pharmacogn Mag., 2015; 11(44):707-715.

Although, curcumin is widely used as a hepatoprotective agent, it is reported to have low bioavailability. Thus, there still exist an unmet industrial need to find a natural agent that is very effective in the management of drug and alcohol induced hepatocellular damage.

Calebin A, another constituent of the rhizomes of Curcuma sp., is known to protect neuronal cells from β-amyloid insult (Park S Y et al, J Nat Prod., 2002; 65(9):1227-31). U.S. Pat. No. 9,737,502 B2 (Majeed et. al) discloses the use of Calebin A in the management of high fat-diet induced hepatic steatosis (Non-alcoholic steatoheapatitis NASH). But, the pathophysiology behind NASH is very different from drug and alcohol induced hepatocellular damage and warrants different treatment modalities. Hence, the present invention discloses a novel and non-obvious use of Calebin A in the management of drug and alcohol induced liver damage and related complications.

The principal object of the invention is to disclose the use of Calebin A for the therapeutic management of drug and alcohol induced hepatocellular damage.

It is another object of the invention to disclose the use of Calebin A for the therapeutic management hyperglycemia associated with drug and alcohol induced hepatocellular damage, by increasing glucose uptake.

The present invention solves the above mentioned objectives and provides further related advantages.

### SUMMARY OF THE INVENTION

The invention discloses a novel use of Calebin A in the prevention of hepatocellular damage. More specifically, the invention discloses the use of Calebin A in mitigating hepatocellular damage in drug and alcohol induced liver toxicity. The invention further discloses a method for the management of hyperglycemia, associated with drug and alcohol induced liver damage by increasing glucose uptake using Calebin A or a composition comprising Calebin A.

Other features and advantages of the present invention will become apparent from the following more detailed description, which illustrate, by way of example, the principle of the invention.

### BRIEF DESCRIPTION OF DRAWINGS

FIG. 1 shows the graphical representation of hepatoprotection activity of Calebin A against alcohol induced toxicity

FIG. 2 shows the graphical representation of hepatoprotection activity of Calebin A against drug (acetaminophen) induced toxicity

FIG. 3 shows the graphical representation of increase in glucose uptake by adipocytes and muscle cells

### DESCRIPTION OF THE MOST PREFERRED EMBODIMENTS

In the most preferred embodiment, present invention relates to a method for protecting hepatic cells from drug and alcohol induced damage, said method comprising step of bringing into contact mammalian hepatic cells with an effective dose of Calebin A, to prevent hepatocellular damage. In related embodiment, the drug is selected from class comprising anaesthetics, non-steroidal anti-inflammatory drugs, anti cancer drugs, antibiotics, anti tuberculosis agents, antiretrovirals and cardiac medications. In another related embodiment the drug preferably belongs to the class of non-steroidal anti-inflammatory drugs. In yet another preferred embodiment, the drug is preferably acetaminophen. In another preferred embodiment, the effective dose of Calebin A is 5-50 μM. In another related embodiment the mammalian cells are human cells.

In another most preferred embodiment, the invention discloses a method of increasing glucose uptake by mammalian cells, said method comprising steps of bringing into contact mammalian cells with effective dose of calebin A, to increase glucose uptake by the cells. In a related embodiment, the increase in glucose uptake by the cells results in the normalisation of blood glucose levels in hyperglycemic conditions. In another preferred embodiment, the hyperglycemic conditions are present in conditions selected from the group comprising liver damage, diabetes, obesity, hyperlipoproteinemia, hyperlipidemia, cardiovascular complications, cancer, atherosclerosis, neurodegenerative diseases, allergy, inflammation, osteoporosis and thyroid dysfunction. In a related embodiment, hyperglycemic condition is preferably associated with liver damage. In another related embodiment, the liver damage is preferably induced by drugs and alcohol. In related embodiment, the drug is selected from class comprising anaesthetics, non-steroidal anti-inflammatory drugs, anti cancer drugs, antibiotics, anti tuberculosis agents, antiretrovirals, and cardiac medications. In another related embodiment the drug preferably belongs to the class of non-steroidal anti-inflammatory drugs. In yet another preferred embodiment, the drug is preferably acetaminophen. In another preferred embodiment, the effective dose of Calebin A is 5-50 μM. In another related embodiment the mammalian cells are human cells.

In another preferred embodiment, the invention discloses a method for the therapeutic management of hyperglycemia in mammals, said method comprising steps of administering effective dose of calebin A, to bring about a reduction in the levels of glucose in the blood. In a related embodiment, the management of hyperglycemia is brought about by increasing cellular uptake of glucose. In another preferred embodiment, hyperglycemia is present in conditions selected from the group comprising liver damage, diabetes, obesity, hyperlipoproteinemia, hyperlipidemia, cardiovascular complications, cancer, atherosclerosis, neurodegenerative diseases, allergy, inflammation, osteoporosis and thyroid dysfunction. In a related embodiment, hyperglycemia is preferably associated with liver damage. In another related embodiment, the liver damage is preferably induced by drugs and alcohol. In related embodiment, the drug is selected from class comprising anaesthetics, non-steroidal anti-inflammatory drugs, anti cancer drugs, antibiotics, anti tuberculosis agents, antiretrovirals, and cardiac medications. In another related embodiment the drug preferably belongs to the class of non-steroidal anti-inflammatory drugs. In yet another preferred embodiment, the drug is preferably acetaminophen. In another preferred embodiment, the effective dose of Calebin A is 5-50 μM. In another related embodiment the mammalian cells are human cells. In another related embodiment calebin A is formulated with pharmaceutically/nutraceutically acceptable excipients, adjuvants, bases, diluents, carriers, conditioning agents, bioavailability enhancers, antioxidants and preservatives and/or combined with other hepatoprotective compositions and administered orally in form of tablets, capsules, syrups, gummies, powders, suspensions, emulsions, chewables, candies or eatables.

The specific examples included herein below illustrate the aforesaid most preferred embodiments of the present invention.

### EXAMPLES

### Example 1: Hepatoprotective Activity

a. Protection from Alcohol Induced Toxicity

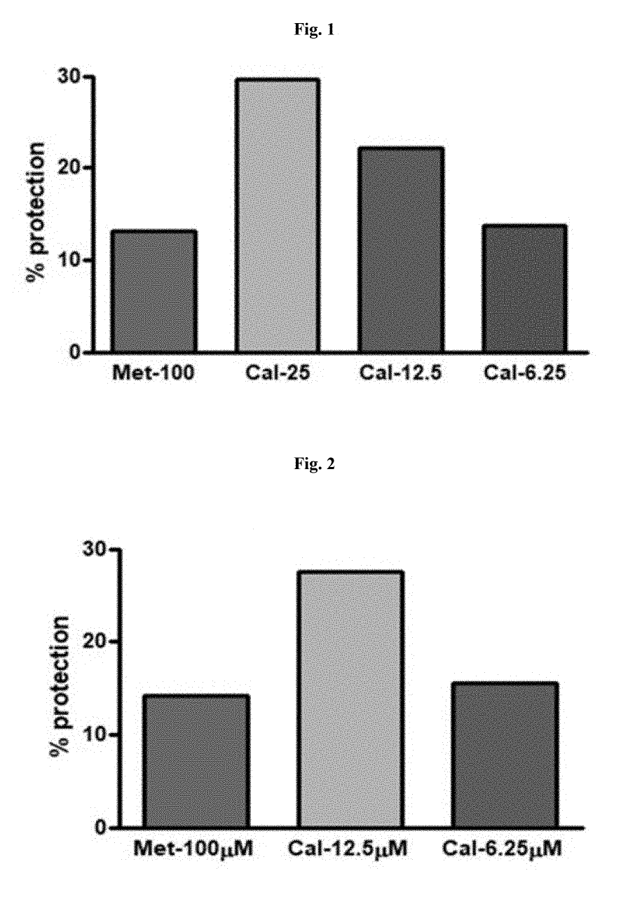

Human HepG2 cells (procured from NCCS, Pune) were cultured in Dulbecco's Modified Eagle Medium (DMEM) supplemented with 10% fetal bovine serum (FBS), 40 μgml⁻¹ gentamycin at 37° C. in an atmosphere of 5% CO₂. When the cells reached 80-90% confluence, they were trypsinized and plated at 5000 cells per well in a 96-well microplate. After 24 hours of seeding, alcohol at a concentration of 1000 mM was added along with different non cytotoxic concentrations of Calebin. Sulphorhodamine B assay was performed after 72 hours of treatment to determine the number of viable cells and percentage protection was calculated (FIG. 1).

Calebin-A at concentration of 6.25 μM protects liver cells (HepG2 cells) from alcohol induced toxicity conferring about 14% protection. Metformin at much higher concentration (100 μM) shows similar protection showing that Calebin A is more effective for preventing alcohol induced hepatocellular toxicity. Progressively higher concentrations of Calebin A (6.25 and 12.5 μM) are able to bring about 23% and 30% protection respectively against alcohol induced hepatocellular damage.

b. Protection from Acetaminophen (Paracetamol) Induced Toxicity

Human HepG2 cells (procured from NCCS, Pune) were cultured in Dulbecco's Modified Eagle Medium (DMEM) supplemented with 10% fetal bovine serum (FBS), 40 μgml⁻¹ gentamycin at 37° C. in an atmosphere of 5% CO₂. When the cells reached 80-90% confluence, they were trypsinized and plated at 5000 cells per well in a 96-well microplate. After 24 hours of seeding, acetaminophen at a concentration of 4 mM was added along with different non cytotoxic concentrations of Calebin. Sulphorhodamine B assay was performed after 72 hours of treatment to determine the number of viable cells and percentage protection was calculated (FIG. 2).

Calebin-A at concentration of 6.25 μM shows superior protection (about 15% protection) against acetaminophen induced toxicity in HepG2 cells when compared to Metformin which brings about 13% protection at much higher concentration (approximately 16 times higher) concentration than Calebin A.

### Example 2: Glucose Uptake Assay

Hyperglycemia is reported to be associated with liver dysfunction (Taub et al., Liver dysfunction hyperglycemia: its etiology and relation to diabetes mellitus, Ann Intern Med. 1945; 22(6):852-862). Type 2 Diabetes, which develops as a complication of liver damage, is also known as “hepatogenous diabetes”. Insulin resistance in muscular and adipose tissues is the pathophysiologic bases of diabetes in liver disease. Liver and skeletal muscles are the two organs mainly involved in maintaining glucose metabolism. Loss of liver function leads to deterioration of skeletal muscles and insulin stimulated glucose uptake is impaired in patients with liver injury (Kruszynska et al., Relationship between insulin sensitivity, insulin secretion and glucose tolerance in cirrhosis, Hepatology., 1991; 14(1):103-11.)

The present invention is aimed at the management of liver damage and its related complications like hyperglycemia using Calebin A.

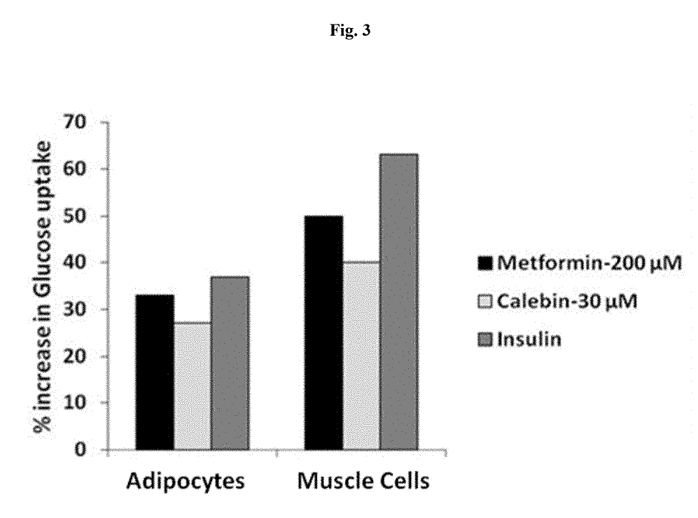

The skeletal muscle cell line C2C12 myoblasts (procured from ATCC) were maintained in DMEM supplemented with 10% Fetal Bovine Serum at 37° C. with 5% CO2. Twenty thousand cells per well were seeded in a 24 well plate. When the cells reached 80-90% confluence, differentiation was induced by replacing the growth medium with DMEM containing 1% horse serum. Experiments were performed in completely differentiated C2C12 myotubes after 4-5 days in differentiation medium. Cells were then treated with 0.5% BSA in low glucose media for 16 hours and washed with cold Krebs-Ringer phosphate buffer without glucose. Cells were then treated with different non cytotoxic concentrations of samples in low glucose DMEM media with or without insulin at a concentration of 0.1 μM for 30 minutes at 37° C. Cells were then washed with cold PBS and stained with 5 μM of a fluorescent D-glucose analog 2-[N-(7-nitrobenz-2-oxa-1,3-diazol-4-yl) amino]-2-deoxy-D-glucose (2-NBDG) for 15 minutes in dark followed by flow cytometric detection of fluorescence produced by the cells (FIG. 3)

Calebin A at lower concentration (3004) is able to increase glucose uptake in adipocytes and muscle cells almost as effective as Metformin in much higher concentration (200 μM-7 times higher concentration as Calebin A)

### Example 3: Formulations Containing Calebin a for Hepatoprotection

Calebin A is formulated with pharmaceutically/nutraceutically acceptable compositions with excipients, adjuvants, bases, diluents, carriers, conditioning agents, bioavailability enhancers, antioxidants and preservatives and/or combined with other hepatoprotective compositions and administered orally in form of tablets, capsules, syrups, gummies, powders, suspensions, emulsions, chewables, candies or eatables and administered for treatment of alcohol induced and drug hepatotoxicity. The following tables provide examples of different Calebin A compositions.

TABLE 1
Calebin A Tablet

Active Ingredients
Calebin A
Excipients
Microcrystalline cellulose, Colloidal silicon dioxide, Magnesium stearate

TABLE 2
Calebin A Capsule

Active Ingredients
Calebin A
Excipients
Microcrystalline cellulose

TABLE 3
Calebin A Tablet

Active Ingredients
Calebin A
Curcumin C³ Complex ®
Excipients
Microcrystalline cellulose, Colloidal silicon dioxide, Magnesium stearate

The above formulations are merely illustrative examples; any formulation containing the above active ingredient intended for the said purpose will be considered equivalent.

Other modifications and variations to the invention will be apparent to those skilled in the art from the foregoing disclosure and teachings. Thus, while only certain embodiments of the invention have been specifically described herein, it will be apparent that numerous modifications may be made thereto without departing from the spirit and scope of the invention. The scope of the invention is to be interpreted only in conjunction with the appended claims.

We claim:

1. A method for protecting hepatic cells from drug and alcohol induced damage, said method comprising step of bringing into contact mammalian hepatic cells with an effective dose of Calebin A, to prevent hepatocellular damage.

2. The method as in claim 1, wherein the drug is selected from class comprising anaesthetics, non-steroidal anti-inflammatory drugs, anticancer drugs, antibiotics, anti tuberculosis agents, antiretrovirals, and cardiac medications.

3. The method as in claim 1, wherein the drug preferably belongs to the class of non-steroidal anti-inflammatory drugs.

4. The method as in claim 1, wherein the drug is preferably acetaminophen.

5. The method as in claim 1, wherein the effective dose of Calebin A is 5-50 μM.

6. The method as in claim 1, wherein the mammalian cells are human cells.

7. A method of increasing glucose uptake by mammalian cells, said method comprising steps of bringing into contact mammalian cells with effective dose of calebin A, to increase glucose uptake by the cells.

8. The method as in claim 7, wherein the increase in glucose uptake by the cells results in the normalisation of blood glucose levels in hyperglycemic conditions.

9. The method as in claim 7, wherein the hyperglycemia is present in disease conditions selected from the group comprising liver damage, diabetes, obesity, hyperlipoproteinemia, hyperlipidemia, cardiovascular complications, cancer, atherosclerosis, neurodegenerative diseases, allergy, inflammation, osteoporosis and thyroid dysfunction.

10. The method as in claim 7, wherein hyperglycemia is preferably associated with liver damage.

11. The method as in claim 7, wherein the liver damage is preferably induced by drugs and alcohol.

12. The method as in claim 7, wherein the drug is selected from class comprising anaesthetics, non-steroidal anti-inflammatory drugs, anticancer drugs, antibiotics, anti tuberculosis agents, antiretrovirals, and cardiac medications.

13. The method as in claim 7, wherein the drug preferably belongs to the class of non-steroidal anti-inflammatory drugs.

14. The method as in claim 7, wherein the drug is preferably acetaminophen.

15. The method as in claim 7, wherein the effective dose of Calebin A is 5-50 μM.

16. The method as in claim 7, wherein the mammalian cells are human cells.

17. A method for the therapeutic management of hyperglycemia in mammals, said method comprising steps of administering effective dose of calebin A, to bring about a reduction in the levels of glucose in blood.

18. The method as in claim 17, wherein the management of hyperglycemia is brought about by increasing cellular uptake of glucose.

19. The method as in claim 17, wherein hyperglycemia is present in conditions selected from the group comprising liver damage, diabetes, obesity, hyperlipoproteinemia, hyperlipidemia, cardiovascular complications, cancer, atherosclerosis, neurodegenerative diseases, allergy, inflammation, osteoporosis and thyroid dysfunction.

20. The method as in claim 17, wherein hyperglycemia is preferably associated with liver damage.

21. The method as in claim 17, wherein the effective dose of Calebin A is 5-50 μM.

22. The method as in claim 17, wherein calebin A is formulated with pharmaceutically/nutraceutically acceptable excipients, adjuvants, bases, diluents, carriers, conditioning agents, bioavailability enhancers, antioxidants and preservatives and/or combined with other hepatoprotective compositions and administered orally in form of tablets, capsules, syrups, gummies, powders, suspensions, emulsions, chewables, candies or eatables.
